(19) United States
(12) Patent Application Publication
(10) Pub. No.: US 2006/0223087 A1
(43) Pub. Date: Oct. 5, 2006

(54) Method for nucleic acid analysis

(76) Inventors: Kiyomi Taniguchi, Kokubunji (JP); Keiichi Nagai, Higashiyamato (JP); Hiroko Matsunaga, Koganei (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/342,861

(22) Filed: Jan. 31, 2006

(30) Foreign Application Priority Data
Mar. 30, 2005 (JP) 2005-098398

(51) Int. Cl. C12Q 1/68

(52) U.S. Cl. 435/6

(57) ABSTRACT

A simple and highly accurate method for detecting the presence or absence of gene mutation and methylated cytosine in CpG dinucleotide that are contained in a target sequence derived from an analysis sample is provided. Features of the method for nucleic acid analysis include cleaving one or more noncomplementary sites in a double-stranded nucleic acid sample by a single strand-specific endonuclease, hybridizing at least one of the nucleic acid fragments obtained to a probe containing a nucleotide sequence that is partially or totally identical to either one strand of the double-stranded nucleic acid sample, allowing an extension reaction to proceed from the nucleic acid fragment hybridized to the probe, and optically detecting pyrophosphate generated by the extension reaction, thereby judging the presence or absence of at least a noncomplementary site in the double-stranded nucleic acid sample.

### CLAIM OF PRIORITY

The present application claims priority from Japanese application JP 2005-098398 filed on Mar. 30, 2005, the content of which is hereby incorporated by reference into this application.

### FIELD OF THE INVENTION

The present invention relates to a method for detecting the presence or absence of various mutations including methylated cytosine in CpG dinucleotide in a target nucleic acid.

### BACKGROUND OF THE INVENTION

DNA methylation in eukaryotic cells occurs at cytosine on the 5′ side of guanine (hereinafter, referred to as “CpG dinucleotide”) in many cases. In particular, a plurality of CpG nucleotides are found in the promoter region of many genes, which is called CpG island. The methylation pattern observed in cancerous cells includes a decrease in methylation over a wide range and high methylation specific to a region (promotor region, etc.). It has been reported that low methylation results in chromosomal instability and an increase in the risk of gene mutation since the expression of unnecessary genes in each cell is not suppressed (refer to Non-patent Document 1: Chen R Z. et al., Nature. 395, p89-93 (1998)). It has also been reported that local hypermethylation is a phenomenon in which intensive methylation is observed in the region involved in gene expression such as promoter region of gene and prevents expression of tumor suppressor genes and DNA repair genes (refer to Non-patent Document 2: Jones P A. et al., Nat. Rev. Genet. 3, p415-428 (2002)).

When frequency of mutation of p16 and the like or frequency of methylation of tumor suppressor genes that are inactivated by methylation of CpG dinucleotides in certain cancers was studied, there were some cases where the frequency observed for abnormal methylation of CpG dinucleotide was higher compared with that for mutations (refer to Non-patent Document 3: Esteller M. et al., Cancer Res. 61, p3225-3229 (2001)). Abnormal methylation of CpG dinucleotide is sometimes detected from an early stage of a precancerous lesion, etc. For example, in lung cancer, it has also been reported that frequency of abnormal methylation of CpG dinucleotide in p16 increases with the progress of the lesion as evidenced by hyperplasia (17%), metaplasia (24%), and carcinoma in situ (50%) (refer to Non-patent Document 4: Belinsky S A. et al., Proc. Natl. Acad. Sci. USA. 95, p11891-11896 (1998)).

Unmethylated cytosine is readily converted to uracil by the bisulfite deamination reaction (refer to Non-patent Document 5: Shapiro R. et al., J. Am. Chem. Soc. 96, p906-912 (1974)). On the other hand, methylated cytosine is not converted to uracil even when treated with bisulfite. Accordingly, the treatment of DNA derived from an analysis sample with bisulfite results in a difference between nucleotide sequences of methylated DNA and unmethylated DNA. The presence or absence of methylation can be detected by taking advantage of this difference.

The method to detect methylation of specific CpG dinucleotides includes a method of methylation specific PCR (MSP) in which detection can be performed at a high sensitivity after treating DNA derived from an analysis sample with bisulfite. However, many primers are required to examine the methylation of CpG dinucleotides over a wide range (refer to Non-patent Document 6: Chan EC. et al., Clin. Cancer Res. 8, p3741-3746 (2002)).

Further, the method to detect methylated cytosine in CpG dinucleotide includes a combined bisulfite restriction analysis (COBRA) method in which DNA derived from an analysis sample after treatment with bisulfite is treated with a restriction enzyme and the presence or absence of methylated cytosine is detected from the result of the fragment analysis (refer to Non-patent Document 7: Xiong Z. et al., Nucleic Acids Res. 25, p2532-2534 (1997)).

Still further, the method to detect methylated cytosine in CpG dinucleotide includes a method in which hybridization is performed between a plurality of capture oligonucleotides immobilized on a substrate and a DNA sample treated with bisulfite that was derived from an analysis sample and then the presence or absence of methylation of cytosine of CpG dinucleotide in the DNA sample is judged from the result (refer to Patent Document 1: JP-A No. 17199/2001).

The method to detect gene mutation includes a reported method in which hybridization is performed between respective PCR amplified products of a DNA sample derived from an analysis sample and a control DNA sample not containing mutation and then a noncomplementary site arising from gene mutation site is cleaved by a single strand-specific nuclease, thereby detecting the presence or absence of gene mutation (Patent Document 2: National Publication of International Patent Application No. 511774/2000).

The method of DNA detection that does not use laser-excited fluorescence includes a method in which pyrophosphate generated correspondingly to the length of nucleotides extended by complementary strand synthesis is converted to ATP, followed by its reaction with luciferin in the presence of luciferase to induce bioluminescence for use in the detection. The reported methods that make use of this technique include a pyrosequencing method for determining DNA nucleotide sequence (Non-patent Document 8: Ahmadian A. et al., Anal. Biochem. 280, p 103-110 (2000)) and a bioluminometric assay with modified extension reaction method (BAMPER method, refer to Non-patent Document 9: Zhou G. et al., Nucleic Acids Res. 29, e93 (2001)) for determining gene mutation and SNP.

### SUMMARY OF THE INVENTION

The conditions required for gene diagnosis are listed as follows: Judgment result is obtained with high accuracy; the method is simple; expensive reagents and apparatus are unnecessary; and multiple sites can be examined collectively. The object of the present invention is to provide a method in which the presence or absence of gene mutation and methylated cytosine at a single site or multiple sites in a target sequence is determined with ease and high accuracy.

As a result of assiduous research intended to overcome the above problems, the present inventors discovered that, after hybridization was performed between a single-stranded nucleic acid having a target sequence derived from DNA of an analysis sample and a single-stranded nucleic acid having a reference sequence complementary to the target sequence except for one or more mutation sites, one or more noncomplementary base pairs formed only in the case when the one or more mutation sites were present in the DNA of the analysis sample were cleaved with a single strand-specific endonuclease, and the presence or absence of one or more mutation sites could be determined by judging whether an extension reaction from at least a fragment generated at the time of cleavage proceeded or not using a nucleic acid probe having a sequence that is partially or totally complementary to the target sequence or the reference sequence as a template.

In other words, the present invention provides a method for analyzing one or more noncomplementary sites in a double-stranded nucleic acid sample including the steps of obtaining nucleic acid fragments by cleaving the one or more noncomplementary sites with a single strand-specific endonuclease;

hybridizing at least one of the obtained fragments to a probe having a nucleotide sequence that is partially or totally identical to either one strand of the double-stranded nucleic acid sample;

performing an extension reaction from the nucleic acid fragment hybridized to the probe; and

optically detecting pyrophosphate generated by the extension reaction.

In the above method, it is desirable that the double-stranded nucleic acid sample is a nucleic acid sample obtained beforehand by amplification of a sequence containing the one or more noncomplementary sites on a target nucleic acid (target gene) that is an analysis target. The length of a region to be amplified is desirably a length of approximately 100 nucleotides to 300 nucleotides containing the one or more noncomplementary sites. If possible, it is desirable that the region to be amplified is designed so that one or more noncomplementary sites that are the analysis target may be located at least 20 nucleotides away from its 5′ end toward the 3′ side. This is because when designed in this range, it is expected that an extension reaction from a nucleic acid fragment generated by the subsequent enzyme cleavage proceeds smoothly.

In the method for nucleic acid analysis of the present invention, one strand of the double-stranded nucleic acid sample is labeled with biotin (for example, using a biotin-labeled primer) in the step of amplification, and a nucleic acid fragment labeled with biotin that is generated by cleavage of the double-stranded nucleic acid having this label with a single strand-specific endonuclease may be recovered as a single-stranded fragment with the use of an avidin-immobilized carrier.

Further, the probe may be immobilized on a solid phase carrier in advance. The solid phase carriers that can be used are, for example, bead, and Sepharose in addition to metal or glass substrate.

The noncomplementary site that is an analysis target includes any mutation site in a double-stranded nucleic acid sample, for example, SNP, microsatellite polymorphism, VNTR, and further, deletion, substitution, and insertion of a specific nucleotide and a site where modification such as methylation is present. Most of all, the method of the present invention is preferably used for analysis of methylated cytosine present in CpG dinucleotide.

When used for analysis of methylated cytosine, it is preferred to convert unmethylated cytosines to thymines by treating the double-stranded nucleic acid sample with bisulfite in advance before the amplification step.

The step of hybridizing a nucleic acid fragment to the probe is preferably carried out by slowly lowering the temperature at a rate not faster than 0.1 degree C./sec to avoid nonspecific hybridization.

In the present invention, the step of optically detecting pyrophosphate can be performed, for example, by a bioluminescence reaction that makes use of luciferin-luciferase. Without being limited to this method, the presence or absence of an extension reaction can be detected by the use of ddNTP labeled with TAMRA, Texas Red, and the like.

The single strand-specific nuclease to be used includes, for example, CELI nuclease, mung bean nuclease, S1 nuclease, and P1 nuclease. As to the nucleic acid fragment after treatment with an enzyme, the fragment end is converted to a blunt end as necessary depending on the characteristic of the enzyme used (for example, CELI).

When there are multiple methylation/mutation sites in one genomic DNA (nucleic acid sample), the cleavage treatment gives rise to fragments of various lengths. When these are detected by a conventional electrophoresis method, signal intensities vary because the length of each fragment is different, resulting in lowering of detection sensitivity. According to the present invention, luminescence intensities to be obtained are the same regardless of the length of each fragment, resulting in improvement of detection sensitivity. Furthermore, it is unnecessary to prepare many primers and probes for every target sequence, and therefore, mutation sites can be easily detected over a wide range.

### BRIEF DESCRIPTION OF THE DRAWINGS

FIG. 1 is a schematic diagram showing the step of treating a methylated DNA or an unmethylated DNA with bisulfite, where FIG. 1A shows that methylated cytosine nucleotide represented by *C is not modified by this chemical treatment and FIG. 1B shows that unmethylated cytosine is converted to uracil;

FIG. 2 is a schematic diagram showing the step of amplifying the methylated DNA or the unmethylated DNA by PCR, where FIG. 2A shows that PCR amplification of the methylated DNA allows methylated cytosine to be amplified as ordinary cytosine and FIG. 2B shows that PCR amplification of the unmethylated DNA after treatment with bisulfite produces a PCR product in which unmethylated cytosine has been converted to thymine;

FIG. 3 is a schematic diagram showing the step of hybridizing a reference sequence that is an oligonucleotide or the PCR product and that is labeled with biotin at the 5′ end and complementary to a target sequence except for methylated cytosine where FIG. 3A shows that the double strand DNA is from the methylated DNA and the reference sequence, giving rise to noncomplementary site, and FIG. 3B shows that the double strand DNA is from the unmethylated DNA and the reference sequence.

FIG. 4 is a schematic diagram showing the step of treating a double strand with a single strand-specific nuclease, where FIG. 4A shows that the noncomplementary site is cleaved by this treatment and FIG. 4B shows that the complementary site remains intact;

FIG. 5 is a schematic diagram showing the step of capturing the biotin-labeled reference sequence by an avidin-immobilized carrier, where FIG. 5A shows that a biotin-labeled fragment generated by the enzyme treatment is captured and FIG. 5B shows that the biotin-labeled intact sequence is captured;

FIG. 6 is a schematic diagram showing the step of hybridization of a biotin-labeled fragment of reference sequence and an oligonucleotide or PCR product containing the sequence complementary to the reference sequence and subsequent extension reaction, where FIG. 6A shows that the biotin-labeled fragment is used for the hybridization and extension reaction and FIG. 6B shows that the biotin-labeled reference sequence is intact.

FIG. 7 is a graph showing the result of detection of luminescence generated by adding a luminescence reagent to a solution after the extension reaction, where FIG. 7A shows the presence of methylated cytosine and FIG. 7B shows the absence of methylated cytosine;

FIG. 8 is a schematic diagram showing the step of hybridizing a reference sequence that is an oligonucleotide or PCR product and that is unlabeled with biotin and complementary to the target sequence except for methylated cytosine where FIG. 8A shows that the double strand DNA is from the methylated DNA and the reference sequence, giving rise to noncomplementary site, and FIG. 8B shows that the double strand DNA is from the unmethylated DNA and the reference sequence.

FIG. 9 is a schematic diagram showing the step of treating a double strand with the single strand-specific nuclease, where FIG. 9A shows that the noncomplementary site is cleaved by this treatment and FIG. 9B shows that the complementary site remains intact;

FIG. 10 is a schematic diagram showing the step of hybridization of a fragment generated by the enzyme treatment to an oligonucleotide or PCR product complementary to the reference sequence and subsequent extension reaction, where FIG. 10A shows that the fragment is hybridized and extended, though hybridization occurs with a plurality of generated fragments because no purification of the fragments was preformed but extension reactions are allowed to proceed with only part of these combinations, and FIG. 10B shows that no extension occurs due to perfect hybridization;

FIG. 11 is a schematic diagram showing the step of hybridizing an reference sequence immobilized on a substrate to a purified single-stranded PCR product, where FIG. 11A shows that the single-stranded PCR product is from a methylated DNA and FIG. 11B shows that the single-stranded PCR product is from an unmethylated DNA;

FIG. 12 is a schematic diagram showing the step of treating a double strand with the single strand-specific nuclease, where FIG. 12A shows that noncomplementary site is cleaved by this treatment and FIG. 12B shows that complementary site remains intact;

FIG. 13 is a schematic diagram showing the step of dehybridizing the nuclease-treated double strand, where FIG. 13A shows that the immobilized oligonucleotide was fragmented and FIG. 13B shows that the immobilized oligonucleotide remained intact;

FIG. 14 is a schematic diagram showing the step of hybridization between the immobilized oligonucleotide and the oligonucleotide or PCR product of the reference sequence and subsequent extension reaction, where FIG. 14A shows an occurrence of the extension reaction for the methylated DNA and FIG. 14B shows no occurrence of the extension reaction for the unmethylated DNA; and

FIG. 15 is a conceptual diagram showing comparison between an electrophoresis method and the method of the present invention when multiple mutation sites are analyzed.

### DETAILED DESCRIPTION OF THE PREFERRED EMBODIMENTS

In the method for gene analysis of the present invention, hybridization is performed between a nucleic acid having a target sequence derived from DNA of an analysis sample and a nucleic acid having a reference sequence complementary to the target sequence except for one or more gene mutations or methylated cytosines, and then, one or more noncomplementary base pairs produced when one or more mutated nucleotides or methylated cytosines are present in the DNA of the analysis sample are cleaved by an enzyme having a single strand-specific endonuclease activity. The presence or absence of one or more gene mutations or methylated cytosines is determined by judging whether an extension reaction from at least one of the fragments generated at the time of cleavage proceeds or not using a nucleic acid having a sequence that is partially or totally complementary to the target sequence or the reference sequence as a template.

When gene mutation is detected, a sequence complementary to the target sequence except for one or more mutated nucleotides in the gene is used for the above reference sequence that is hybridized to the target sequence derived from the DNA of the analysis sample.

When one or more methylated cytosines are detected as described above, unmethylated cytosines contained in the DNA of the analysis sample are subjected beforehand to treatment with bisulfite to convert them to uracils by deamination.

For the above reference sequence used to hybridize to the target sequence derived from the DNA of the analysis sample when one or more methylated cytosines are detected, a sequence complementary to the nucleotide sequence in which all cytosines except for those of CpG dinucleotides contained in the target sequence are converted to thymines or a sequence complementary to the nucleotide sequence in which all cytosines contained in the target sequence are converted to thymines is used.

The presence or absence of an extension reaction can be judged by the step of converting pyrophosphate generated by the extension reaction between the above fragment and the above reference sample to ATP and detecting luminescence resulting from its reaction with luciferin in the presence of luciferase.

The extension reaction using the fragment generated from the target sequence or the reference sequence by cleavage at the 3′ side of one or more noncomplementary sites with a single strand-specific endonuclease and a template nucleic acid having the sequence complementary to the target sequence containing no mutation nor methylated cytosine or the sequence complementary to the reference sequence may be carried out either in a solution or by the use of a solid carrier immobilized with an oligonucleotide having the reference sequence described above.

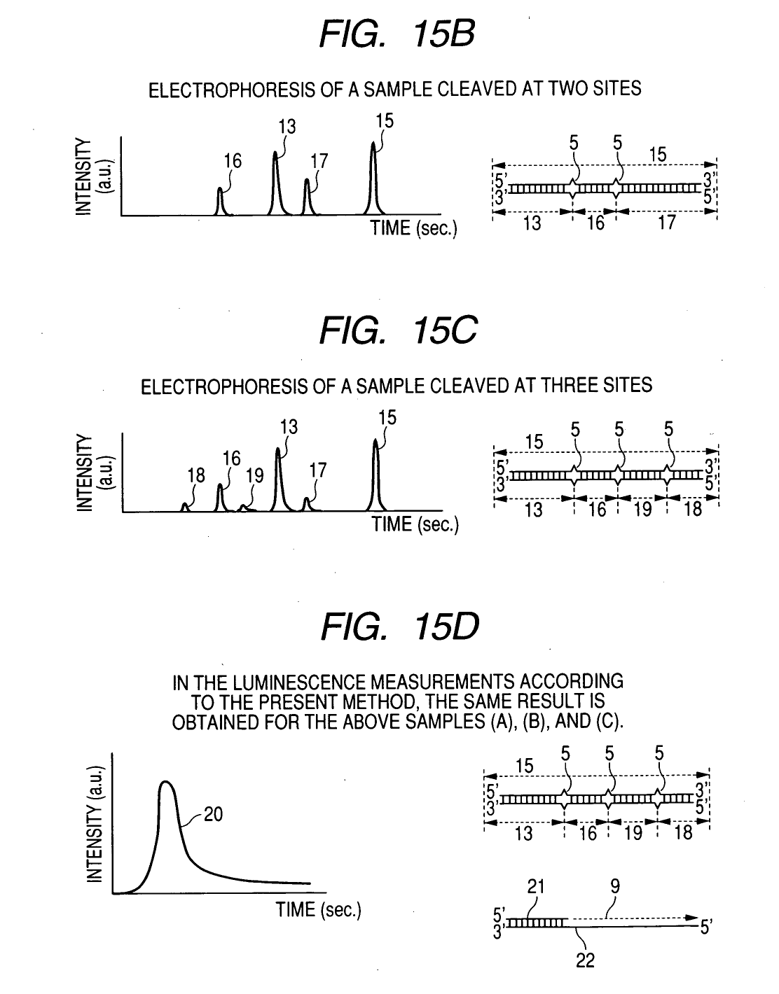

A comparison (conception) between an electrophoresis method and the method of the present invention when multiple mutation sites are analyzed is shown in FIG. 15. As is apparent from FIG. 15, when there are multiple methylation or mutation sites in one genomic DNA (nucleic acid sample), fragments of various lengths are produced by the cleavage treatment. When these are detected by conventional electrophoresis, signal intensities vary because of the variation in the length of each fragment, resulting in lowering of detection sensitivity. According to the present invention, expected luminescent intensities become the same even if the lengths of the fragments differ, and therefore, the detection sensitivity is enhanced. Furthermore, it is unnecessary to prepare many oligonucleotides needed for every site to be studied and one or more gene mutations or methylated cytosines can be detected over a wide range.

### EXAMPLES

Hereinafter, the present invention is more specifically explained by means of the following examples. These are shown merely as examples of detection of one or more gene mutations and methylations and are not intended to be limiting of the scope of the present invention. As to the oncogene K-Ras used in example 1, a gene mutation was present at the codon 12 in DNA derived from cancer cells used here, and no gene mutation was present in DNA derived from non-cancerous cells. As to three CpG dinucleotide cytosines present in the promoter region of the tumor suppressor gene hMLH1 used in examples 2 to 5, all of these were methylated in DNA derived from cancer cells used here, while all of these were unmethylated in DNA derived from non-cancerous cells.

### Example 1

The presence or absence of gene mutation was determined for a gene mutation at the codon 12 of the oncogene K-Ras. Genomic DNA was extracted from a sample according to a general method (Molecular Cloning Third Edition (2001) pp 6.8-6.11 (Cold Spring Harbor Laboratory Press)). Two microliters of the extracted genomic DNA (20 ng/μl), 10 μM primer (Table I), 2.5 mM dNTP, and a PCR buffer (QIAGEN Inc.) and 0.2 μl of 5 U/μl Taq DNA polymerase (QIAGEN Inc.) were mixed and adjusted to 20 μl with sterile water.

After heat denaturation for 1.5 min at 94 degrees C., PCR amplification was performed with 35 cycles of 94 degrees C. for 30 sec, 55 degrees C. for 30 sec, and 72 degrees C. for 1 min, followed by one cycle of 72 degrees C. for 2 min. The sequences of a PCR product to be amplified and primers are shown in Table I below.

TABLE I

A: PCR primer (The 5′terminus of K-RasF is labeled with biotin)

| | | | | PCR | |
|---|---|---|---|---|---|
| | | Length | Tm | product | |
| Primer | Sequence (5′→3′) | (bp) | (° C.) | (bp) | SEQ ID NO |
| K-RasF | GACTGAATATAAACTTGTGGTAGTTG | 26 | 62.4 | 107 | 1 |
| K-RasR | CTATTGTTGGATCATATTCGTCC | 23 | 63.4 | | 2 |

```
B: Normal sequence of PCR product (SEQ ID NO: 3: Gene mutation
at codon 12: GGT→TGT)
                      10 20 30 40 50 60
5′ GACTGAATAT AAACTTGTGG TAGTTGGAGC TGGTGGCGTA GGCAAGAGTG CCTTGACGAT 3′
3′ CTGACTTATA TTTGAACACC ATCAACCTCG ACCACCGCAT CCGTTCTCAC CGAACTGCTA 5′
                      70 80 90 100 107
5′ ACAGCTAATT CAGAATCATT TTGTGGACGA ATATGATCCA ACAATAG 3′
3′ TGTCGATTAA GTCTTAGTAA AACACCTGCT TATACTAGGT TGTTATC 5′
```

To enhance efficiency of later hybridization, the PCR primers remaining in the double-stranded PCR product and dNTP were removed (QIAquick PCR purification kit: QIAGEN Inc.). To 10 μl of this double-stranded PCR product, 8 μl of streptavidin Sepharose (Amersham Biosciences Ltd.) and 42 μl of a binding buffer (10 mM Tris-HCl (pH 7.5), 2 M NaCl, 1 mM EDTA, 0.01% Tween 20 (w/v)) were added and mixed well for 5 min to allow the double-stranded PCR product labeled with biotin on one of the strands to be captured by streptavidin. This mixture was applied in 60 μl aliquots into each well of a Multi-Screen 96-well plate (Millipore Corp.) and centrifuged (2,450 rpm, 3 min, room temperature). After 50 μl of 0.2 M NaOH was applied into each well to denature the double-stranded PCR product by alkali, followed by centrifugation (2,450 rpm, 3 min, room temperature), the filtrate containing a single-stranded PCR product not labeled with biotin was recovered and submitted to purification by ethanol precipitation.

A solution was prepared by adding 1 μl of the single-stranded PCR product (50 ng nucleic acid) representing the target sequence that was derived from an analysis sample, 1 μl of a single-stranded DNA (50 ng nucleic acid) representing the reference sequence that was complementary to the target sequence except for the mutated nucleotide and labeled with biotin at the 5′ end side, and further 1 μl of a hybridization buffer (100 mM Tris-HCl, 1 M NaCl, 0.5 mM EDTA) and adjusting to 8 μl with sterile water.

Target sequence: (The underlined nucleotide is the mutation site)

(SEQ ID NO: 4)

3′-CTGACTTATATTTGAACACCATCAACCTCGAACACCGCATCCGTTCT
CACGGAACTGCTATGTCGATTAAGTCTTAGTAAAACACCTGCTTATACTA
GGTTGTTATC-5′

Reference sequence: (The underlined nucleotide corresponds to the mutation site)

(SEQ ID NO: 5)

5′-GACTGAATATAAACTTGTGGTAGTTGGAGCTGGTGGCGTAGGCAAGA
GTGCCTTGACGATACAGCTAATTCAGAATCATTTTGTGGACGAATATGAT
CCAACAATAG-3′

The solution was subjected to heat denaturation for 10 min at 95 degrees C., and hybridization was performed under the condition that the temperature was slowly lowered up to 25 degrees C. at a rate of 0.1 degree C./sec to avoid nonspecific hybridization. When gene mutation was present in the target sequence, a noncomplementary site appeared at the mutated nucleotide.

To 8 μl of the sample (100 ng nucleic acid) that was subjected to hybridization in the above step, 1 μl of 10× buffer solution (100 mM KCl, 100 mM MgSO₄, 100 mM HEPES, 0.02% Triton X100, 0.002 mg/ml BSA) and 0.5 μl each of an enhancer and a single strand-specific nuclease CELI (TRANSGENOMIC, Inc.) were added to prepare a reaction solution of 10 μl in total. This was incubated for 15 min at 45 degrees C. to digest enzymatically the above noncomplementary site, followed by terminating the single strand-specific nuclease reaction by adding 1 μl of 500 mM EDTA. Further, the fragment ends were converted to blunt ends as necessary depending on the characteristic of the enzyme used.

To 10 μl of the biotin-labeled fragment produced from the reference sequence by digesting with the single strand-specific nuclease, 8 μl of streptavidin Sepharose and 42 μl of a binding buffer (10 mM Tris-HCl (pH 7.5), 2 M NaCl, 1 mM EDTA, 0.01% Tween 20 (w/v)) was added and mixed well for 5 min to allow the biotin-labeled fragment to be captured by streptavidin. This 60 μl mixture was applied into Multi-Screen 96-well plate (Millipore Corp.) and centrifuged (2,450 rpm, 3 min, room temperature). The streptavidin Sepharose capturing the single strand fragment (labeled with biotin at the 5′ end) that was present on a membrane of the Multi-Screen 96-well plate was suspended in 10 μl of sterile water and recovered.

To 1.5 μl of the fragment sample recovered in the above step, 1 μl of a single-stranded DNA sample (1 μM) complementary to this sequence, 0.3 μl of 10×Taq buffer (Amersham Biosciences Ltd.), 0.15 μl of 1 mM dNTP (pretreated with pyrophosphatase), and 0.03 μl of 5 U/μl Taq polymerase (Amersham Biosciences Ltd.) were added, mixed, and adjusted to 3 μl with sterile water. After heat denaturation for 2 min at 95 degrees C., an extension reaction was performed for 1 min at 55 degrees C., and then the reaction solution was returned to room temperature.

Single-stranded DNA sample complementary to the fragment sample: (The underlined portion is a site corresponding to the mutation.)

(SEQ ID NO: 6)

3′-CTGACTTATATTTGAACACCATCAACCTCGACCACCGCATCCGTTCT
CACGGAACTGCTATGTCGATTAAGTCTTAGTAAAACACCTGCTTATACTA
GGTTGTTATC-5′

To the extension reaction solution was added 3 μl of the luciferin-luciferase bioluminescence reagent (Zhou G. et al., Nucleic Acids Res. 29, e93 (2001)) to detect luminescence. When the mutation was present in the target sequence, the cleaved fragment allowed an extension reaction to proceed to produce pyrophosphate, and therefore luminescence was observed. However, when no mutation was present in the target sequence, the extension reaction did not proceed and thus pyrophosphate was not produced, resulting in no observation of the luminescent reaction via a series of reactions.

### Example 2

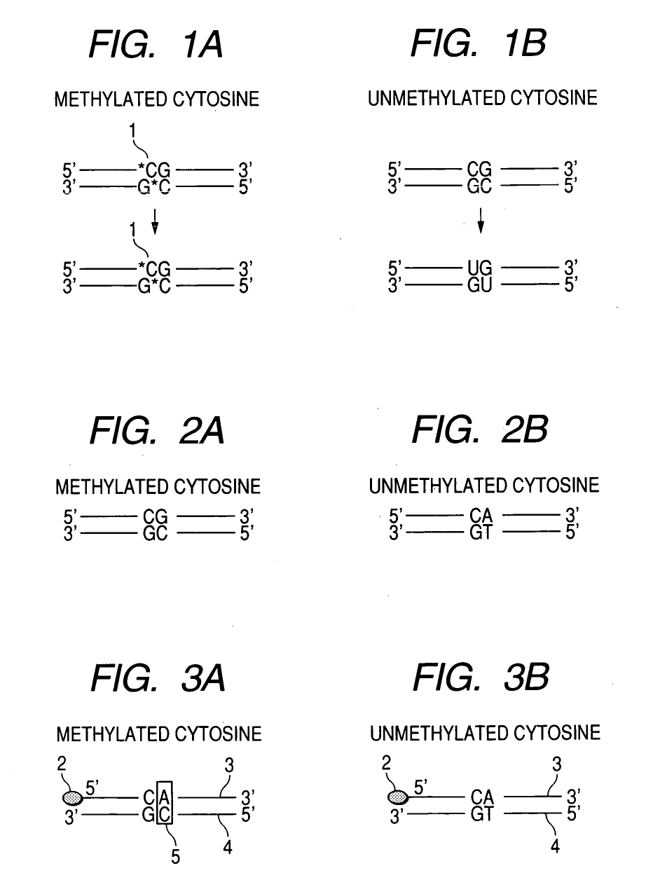

Genomic DNA was extracted from a sample according to the general method (Molecular Cloning Third Edition (2001) pp 6.8-6.11 (Cold Spring Harbor Laboratory Press)). To 50 μl of 20 ng/μl genomic DNA derived from an analysis sample, 5.5 μl of 2 M NaOH was added and incubated for 30 min at 37 degrees C., and then 30 μl of 10 mM hydroquinone and 520 μl of 3 M bisulfite solution were added and incubated overnight (16 to 20 hours) at 50 to 55 degrees C. in the dark. The DNA treated with bisulfite was purified using Wizard DNA purification kit (Promega). By this chemical treatment, unmethylated cytosines in the extracted genomic DNA were converted to uracils (FIG. 1).

The DNA sequence before the treatment with bisulfite and the DNA sequence after the treatment with bisulfite using DNAs containing methylated cytosines and no methylated cytosines in the promoter region of hMLH1 gene are shown in Table II below. The portions underlined in Table II indicate portions where cytosine bases of CpG dinucleotides are present.

TABLE II

```
A: Genomic DNA sequence of promoter region of hMLH1 gene (SEQ ID NO: 7)
                      10 20 30 40 50 60
5′ CAAGCGCATA TCCTTCTAGG TAGCGGGCAG TAGCCGCTTC AGGGAGGGAC GAAGAGACCC 3′
3′ GTTCGCGTAT AGGAAGATCC ATCGCCCGTC ATCGGCGAAG TCCCTCCCTG CTTCTCTGGG 5′
                      70 80 90 100 110 118
5′ AGCAACCCAC AGAGTTGAGA AATTTGACTG GCATTCAAGC TGTCCAATCA ATAGCTGC 3′
3′ TCGTTGGGTG TCTCAACTCT TTAAACTGAC CGTAAGTTCG ACAGGTTAGT TATCGACG 5′
B: DNA sequence of DNA containing methylated cytosines after
bisulfite treatment (SEQ ID NO: 8)
                      10 20 30 40 50 60
5′ UAAGCGUATA TUUTTUTAGG TAGCGGGUAG TAGUCGUTTU AGGGAGGGAC GAAGAGAUUU 3′
3′ GTTUGCGTAT AGGAAGATUU ATUGCUUGTU ATUGGCGAAG TUUUTUUUTG CTTUTUTGGG 5′
                      70 80 90 100 110 118
5′ AGUAAUUUAU AGAGTTGAGA AATTTGAUTG GUATTUAAGU TGTUUAATUA ATAGUTGU 3′
3′ TUGTTGGGTG TUTUAAUTUT TTAAAUTGAU UGTAAGTTUG AUAGGTTAGT TATUGAUG 5′
```

(C: Methylated cytosine)

```
C: DNA sequence of DNA containing no methylated cytosines after
bisulfite treatment (SEQ ID NO: 9)
                      10 20 30 40 50 60
5′ UAAGUGUATA TUUTTUTAGG TAGUGGGUAG TAGUUGUTTU AGGGAGGGAU GAAGAGAUUU 3′
3′ GTTUGUGTAT AGGAAGATUU ATUGUUUGTU ATUGGUGAAG TUUUTUUUTG UTTUTUTGGG 5′
                      70 80 90 100 110 118
5′ AGUAAUUUAU AGAGTTGAGA AATTTGAUTG GUATTUAAGU TGTUUAATUA ATAGUTGU 3′
3′ TUGTTGGGTG TUTUAAUTUT TTAAAUTGAU UGTAAGTTUG AUAGGTTAGT TATUGAUG 5′
```

(U: Unmethylated cytosine)

PCR amplification of the promoter region of hMLH1 gene was carried out using the purified DNA. Two microliters each of the DNAs after treatment with bisulfite, 10 μM each primer (forward direction, reverse direction), 2.5 mM dNTP, and 10×PCR buffer solution (QIAGEN Inc.) and further 0.5 μl of 5 U/μl Taq DNA polymerase (QIAGEN Inc.) were mixed and adjusted to 20 μl with sterile water. After heat denaturation for 1.5 min at 94 degrees C., PCR amplification was performed with 35 cycles of 94 degrees C. for 30 sec, 55 degrees C. for 30 sec, and 72 degrees C. for 1 min, followed by one cycle of 72 degrees C. for 2 min. Since unmethylated cytosines were converted to uracils by the treatment with bisulfite, the PCR product using the DNA after the treatment with bisulfite as a template had a sequence in which uracils were further substituted by thymines (FIG. 2B). On the other hand, since the nucleotide substitution did not occur in methylated cytosines even after treated with bisulfite, the state of being cytosine was retained even when PCR amplification was performed using the DNA treated with bisulfite as a template (FIG. 2A). The sequences of PCR primers are shown in Table IIIA. One of the PCR primers used (hMLH1F) was labeled with biotin at its 5′ end. The sequences of PCR products when amplification was performed using the DNAs containing methylated cytosines and unmethylated cytosines respectively as a template are shown in Table IIIB and Table IIIC.

TABLE III

A: hMLH1 PCR primer

| | | | | PCR | |
|---|---|---|---|---|---|
| | | Length | Tm | product | |
| Primer | Sequence (5′→3′) | (bp) | (° C.) | (bp) | SEQ ID NO |
| hMLH1F | CAAACGCATATCCTTCTAAATAA | 23 | 62.2 | 118 | 10 |
| hMLH1R | GTAGTTATTGATTGGATAGTTTGAAT | 26 | 61.6 | | 11 |

```
B: PCR product using DNA containing methylated cytosines as
template (SEQ ID NO: 12)
                      10 20 30 40 50 60
5′ CAAACGCATA TCCTTCTAAA TAACGAACAA TAACCGCTTC AAAAAAAAAC GAAAAAACCC 3′
3′ GTTTGCGTAT AGGAAGATTT ATTGCTTGTT ATTGGCGAAG TTTTTTTTTG CTTTTTTGGG 5′
                      70 80 90 100 110 118
5′ AACAACCCAC AAAATTAAAA AATTTAACTA ACATTCAAAC TATCCAATCA ATAACTAC 3′
3′ TTGTTGGGTG TTTTAATTTT TTAAATTGAT TGTAAGTTTG ATAGGTTAGT TATTGATG 5′
```

(C: Methylated cytosine)

```
C: PCR product using DNA containing no methylated cytosine as
template (SEQ ID NO: 13)
                      10 20 30 40 50 60
5′ CAAACGCATA TCCTTCTAAA TAACAAACAA TAACCACTTC AAAAAAAAAC AAAAAAACCC 3′
3′ GTTTGCGTAT AGGAAGATTT ATTGTTTGTT ATTGGTGAAG TTTTTTTTTG TTTTTTTGGG 5′
                      70 80 90 100 110 118
5′ AACAACCCAC AAAATTAAAA AATTTAACTA ACATTCAAAC TATCCAATCA ATAACTAC 3′
3′ TTGTTGGGTG TTTTAATTTT TTAAATTGAT TGTAAGTTTG ATAGGTTAGT TATTGATG 5′
```

(T: Methylated cytosine)

To enhance efficiency of later hybridization, the primers remaining in the PCR product and dNTP were removed (QIAquick PCR purification kit: QIAGEN Inc.). To 10 μl of this PCR product, 8 μl of streptavidin Sepharose (Amersham Biosciences Ltd.) and 42 μl of the binding buffer (10 mM Tris-HCl (pH 7.5), 2 M NaCl, 1 mM EDTA, 0.01% Tween 20 (w/v)) were added and mixed well for 5 min to allow the PCR product labeled with biotin to be captured by streptavidin. This mixture was applied in 60 μl aliquots into each well of a Multi-Screen 96-well plate (Millipore Corp.) and centrifuged (2,450 rpm, 3 min, room temperature). After 50 μl of 0.2 M NaOH was further applied into each well and centrifuged (2,450 rpm, 3 min, room temperature), the filtrate containing a single-stranded PCR product (unlabeled with biotin) was recovered and submitted to purification by ethanol precipitation.

The single-stranded PCR product (50 ng nucleic acid) representing a target sequence and the single-stranded DNA (50 ng nucleic acid) representing the reference sequence that was complementary to the sequence in which all cytosines in the target sequence were converted to thymines and was labeled with biotin at its 5′ end were mixed well with each other, and 1 μl of the hybridization buffer (100 mM Tris-HCl, 1 M NaCl, 0.5 mM EDTA) was added. The final volume was adjusted to 8 μl. Target sequence: (The underlined portions are sites corresponding to methylated cytosines)

(SEQ ID NO: 14)

3′-GTTTGCGTATAGGAAGATTTATTGCTTGTTATTGGCGAAGTTTTTTT
TTGCTTTTTTGGGTTGTTGGGTGTTTTAATTTTTTAAATTGATTGTAAGT
TTGATAGGTTAGTTATTGATG-5′

Reference sequence: (The underlined portions are sites corresponding to unmethylated cytosines)

(SEQ ID NO: 15)

5′-CAAACGCATATCCTTCTAAATAACAAACAATAACCACTTCAAAAAAA
AACAAAAAAACCCAACAACCCACAAAATTAAAAAATTTAACTAACATTCA
AACTATCCAATCAATAACTAC-3′

This mixed sample was heat denatured for 10 min at 95 degrees C., and hybridization was performed in the step of lowering the temperature slowly up to 25 degrees C. at a rate of 0.1 degree C./sec to avoid nonspecific hybridization. When methylated cytosines were present in the DNA sample derived from the analysis sample, noncomplementary sites were generated due to inability of complementary binding as shown in FIG. 3.

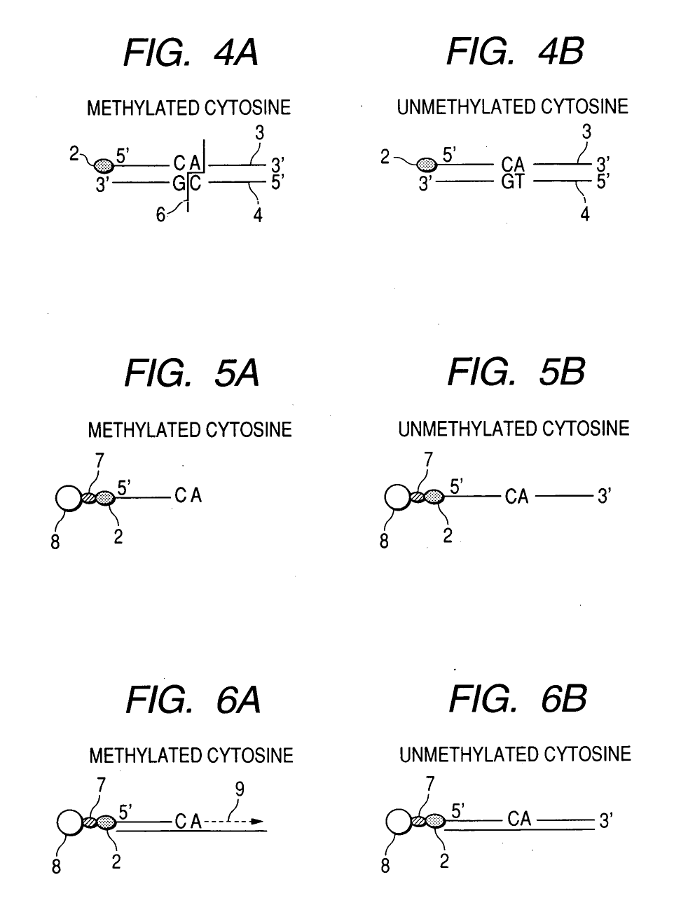

To 8 μl of the sample (100 ng nucleic acid) that was subjected to hybridization in the above step, 1 μl of 10× buffer solution (100 mM KCl, 100 mM MgSO₄, 100 mM HEPES, 0.02% Triton X100, 0.002 mg/ml BSA) and 0.5 μl each of the enhancer and the single strand-specific nuclease CELI (TRANSGENOMIC, Inc.) were added and mixed to prepare a reaction solution of 10 μl in total. This was incubated for 15 min at 45 degrees C. to enzymatically digest the above noncomplementary sites (FIG. 4), followed by terminating the single strand-specific nuclease reaction by adding 1 μl of 500 mM EDTA. Further, the fragment ends were converted to blunt ends as necessary depending on the characteristic of the enzyme used.

To 10 μl of the fragment labeled with biotin that was derived from the reference sequence by the digestion reaction, 8 μl of streptavidin Sepharose and 42 μl of the binding buffer (10 mM Tris-HCl (pH 7.5), 2 M NaCl, 1 mM EDTA, 0.01% Tween 20 (w/v)) were added and mixed well for 5 min to allow the fragment labeled with biotin to be captured by streptavidin. This 60 μl mixture was applied in 60 μl aliquots into each well of a Multi-Screen 96-well plate (Millipore Corp.) and centrifuged (2,450 rpm, 3 min, room temperature). The streptavidin Sepharose capturing the single strand fragment (labeled with biotin at the 5′ end) that was present on the membrane of the Multi-Screen 96-well plate was suspended in 10 μl of sterile water and recovered (FIG. 5).

To the streptavidin Sepharose were added 1 μl of 1 μM single-stranded DNA having a nucleotide sequence (SEQ ID NO:16) complementary to the reference sequence and 0.3 μl of 10×Taq buffer (Amersham Biosciences Ltd.), 0.15 μl of 1 mM dNTP (pretreated with pyrophosphatase), and 0.03 μl of 5 U/μl Taq polymerase (Amersham Biosciences Ltd.), mixed, and adjusted to 3 μl with sterile water. After this was heat denatured for 2 min at 95 degrees C., an extension reaction was performed for 1 min at 55 degrees C. as shown in FIG. 6, and then the reaction solution was returned to room temperature.

Nucleotide sequence complementary to reference sequence:

3′-GTTTGCGTATAGGAAGATTTATTGTTTGTTATTGGTGAAGTTTTTTT
TTGTTTTTTTGGGTTGTTGGGTGTTTTAATTTTTTAAATTGATTGTAAGT
TTGATAGGTTAGTTATTGATG-5′

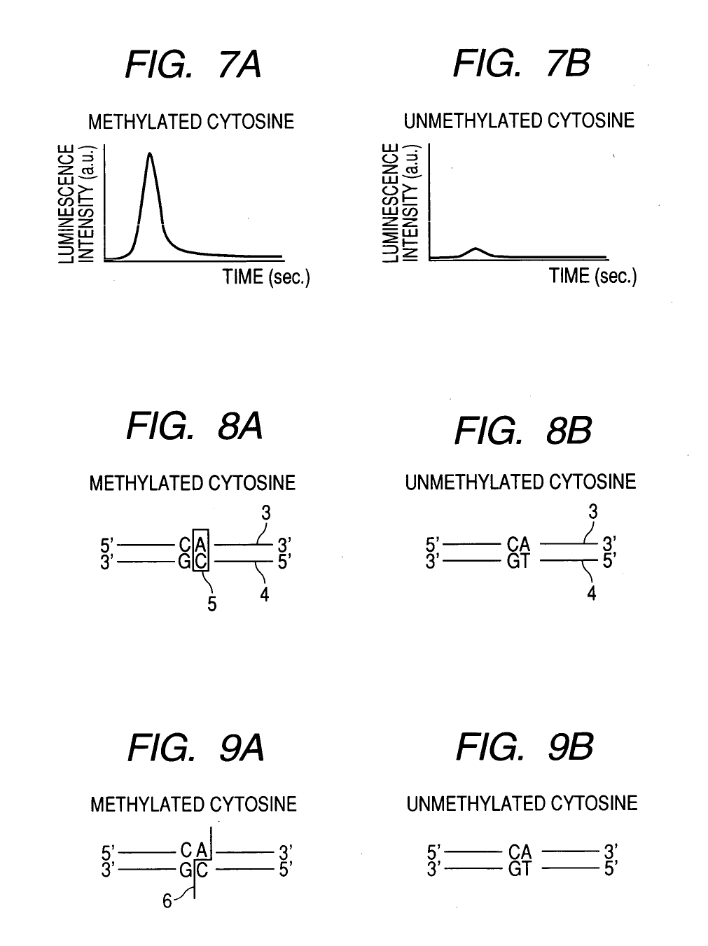

To the extension reaction solution was added 3 μl of the luciferin-luciferase bioluminescence reagent to detect luminescence by making use of pyrophosphate generated by the extension reaction. When methylated cytosines were present in the target sequence, the cleaved fragment allowed the extension reaction to proceed to produce pyrophosphate, and therefore luminescence was observed. However, when no methylated cytosine was present in the target sequence, the extension reaction did not proceed and thus pyrophosphate was not produced, resulting in no observation of the luminescent reaction via a series of reactions (FIG. 7).

### Example 3

In the example 2, when the reference sequence to hybridize to the single-stranded PCR product (50 ng nucleic acid) representing the target sequence was changed to a sequence (SEQ ID NO:17) complementary to the sequence in which all cytosines except for CpG dinucleotides in the target sequence were converted to thymines, only the sample unmethylated on cytosines of CpG dinucleotides produced fragments by the digestion reaction. Therefore, the subsequent extension reaction and luminescence reaction were observed. Namely, it was confirmed that the luminescence was detected when cytosines of CpG dinucleotides were unmethylated, whereas the luminescence reaction was not observed when these were methylated.

Modified reference sequence: (The underlined portions are sites corresponding to methylated cytosines)

(SEQ ID NO: 17)

5′-CAAACGCATATCCTTCTAAATAACGAACAATAACCGCTTCAAAAAAA
AACGAAAAAACCCAACAACCCACAAAATTAAAAAATTTAACTAACATTCA
AACTATCCAATCAATAACTAC-3′

### Example 4

Genomic DNAs were extracted from a sample and normal cells according to the general method (Molecular Cloning Third Edition (2001) pp 6.8-6.11 (Cold Spring Harbor Laboratory Press)). To 50 μl of 20 ng/μl each genomic DNA, 5.5 μl of 2 M NaOH was added and incubated for 30 min at 37 degrees C., and then 30 μl of 10 mM hydroquinone and 520 μl of 3 M bisulfite solution were added and incubated overnight (16 to 20 hours) at 50 to 55 degrees C. in the dark. The DNA treated with bisulfite was purified using Wizard DNA purification kit (Promega Corp.) By this chemical treatment, unmethylated cytosines in the extracted genomic DNAs were converted to uracils.

PCR amplification of the promoter region of hMLH1 gene was performed with the use of the DNAs derived from the sample and the normal cells both of which had been treated with bisulfite. The PCR primers used (SEQ ID NO:10 and SEQ ID NO:11) were both unlabeled with biotin at their 5′ ends. Two microliters each of each DNA after treatment with bisulfite, 10 μM each primer (forward direction, reverse direction), 2.5 mM dNTP, and 10×PCR buffer solution (QIAGEN Inc.) and 0.5 μl of 5 U/μl Taq DNA polymerase (QIAGEN Inc.) were mixed and adjusted to 20 μl with sterile water. After heat denaturation for 1.5 min at 94 degrees C., PCR amplification was performed with 35 cycles of 94 degrees C. for 30 sec, 55 degrees C. for 30 sec, and 72 degrees C. for 1 min, followed by one cycle of 72 degrees C. for 2 min. Remaining primers contained in the PCR product and remaining dNTP were removed with the use of the QIAquick PCR purification kit (QIAGEN Inc.).

The PCR product derived from the sample (50 ng nucleic acid) and the PCR product derived from the normal cells (50 ng nucleic acid) both of which were obtained in the above step were mixed, followed by addition of 1 μl of the hybridization buffer (100 mM Tris-HCl, 1 M NaCl, 0.5 mM EDTA). The final volume was adjusted to 8 μl with sterile water. To destroy their secondary structures, the mixed sample was heat denatured for 10 min at 95 degrees C., and hybridization was performed under the condition that the temperature was slowly lowered up to 25 degrees C. at a rate of 0.1 degree C./sec to avoid nonspecific hybridization. When methylated cytosines were present in the sample, a double strand having noncomplementary sites were generated in part of the mixed sample subjected to the hybridization (FIG. 8).

To 8 μl of the mixed sample (100 ng nucleic acid) that was subjected to hybridization in the above step, 1 μl of 10× buffer solution (100 mM KCl, 100 mM MgSO₄, 100 mM HEPES, 0.02% Triton X100, 0.002 mg/ml BSA) and 0.5 μl each of the enhancer and the single strand-specific nuclease CELI (TRANSGENOMIC, Inc.) were added and mixed to prepare a solution of 10 μl with sterile water. This was incubated for 15 min at 45 degrees C. to enzymatically digest the above noncomplementary sites (FIG. 9), followed by immediate addition of 1 μl of 500 mM EDTA to terminate the single strand-specific nuclease reaction. Further, the fragment ends were converted to blunt ends.

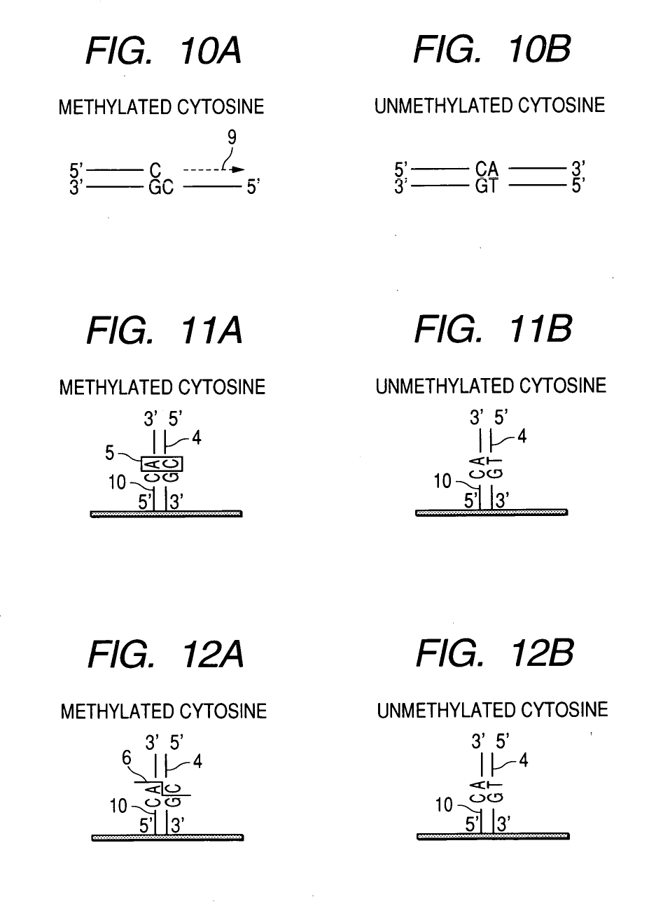

Next, 1.5 μl of the mixed sample enzymatically digested at noncomplementary sites in the above step and 1 μl of 1 μM PCR product amplified using the analysis sample were mixed, added with 0.3 μl of 10×Taq buffer (Amersham Biosciences Ltd.), 0.15 μl of 1 mM dNTP, and 0.03 μl of 5 U/μl Taq polymerase (Amersham Biosciences Ltd.), and adjusted to 3 μl with sterile water. After this was heat denatured for 2 min at 95 degrees C., an extension reaction was performed for 1 min at 55 degrees C. as shown in FIG. 10, and then the reaction solution was returned to room temperature. At this time, since the produced fragments were not purified, hybridization should have occurred with a plurality of the fragments; however extension reactions that were allowed to proceed might have been limited to part of these combinations.

To the extension reaction solution was added 3 μl of the luciferin-luciferase bioluminescence reagent to detect luminescence by making use of pyrophosphate generated by the extension reaction. When methylated cytosines were present in the sample, the cleaved fragment allowed the extension reaction to proceed to produce pyrophosphate, and therefore luminescence was observed. However, when no methylated cytosine was present, the extension reaction did not proceed and thus pyrophosphate was not produced, resulting in no observation of the luminescent reaction via a series of reactions.

### Example 5

A genomic DNA extracted from an analysis sample in a step similar to that in the example 2 was treated with bisulfite. The promoter region of hMLH1 gene was amplified by PCR, followed by submitting to the single strand purification and ethanol precipitation purification. The sequences of the PCR primers are shown in Table III A. One of the primers used (hMLH1F) was labeled with biotin at its 5′ end.

A single-stranded PCR product obtained as a target sequence was adjusted to 3 μl with PerfectHyb^{R} hybridization solution (TOYOBO Co., LTD.), and then the secondary structure was destroyed by heating for 3 min at 95 degrees C. After allowing it to return to room temperature, 3 μl of the sample was applied onto a substrate and hybridized to a probe immobilized on the substrate in advance for 1 hour at 42 degrees C. under the condition of 100% humidity (FIG. 11). The probe used here had a nucleotide sequence complementary to the sequence in which all cytosines in the target sequence were converted to thymines. Then, the substrate was washed twice with sterile water to remove unhybridized remnants.

In the mean time, 0.6 μl of 10× buffer solution (100 mM KCl, 100 mM MgSO₄, 100 mM HEPES, 0.02% Triton X100, 0.002 mg/ml BSA) and 0.5 μl each of the enhancer and the single strand-specific nuclease CELI (TRANSGENOMIC, Inc.) were mixed and adjusted to 6 μl with sterile water. This was applied onto the substrate that had been subjected to hybridization in the above step and incubated for 15 min at 45 degrees C. to digest noncomplementary sites as shown in FIG. 12, followed by immediate addition of 1 μl of 500 mM EDTA to terminate the single strand-specific nuclease reaction. Further, the fragment ends were converted to blunt ends as necessary depending on the characteristic of the enzyme used.

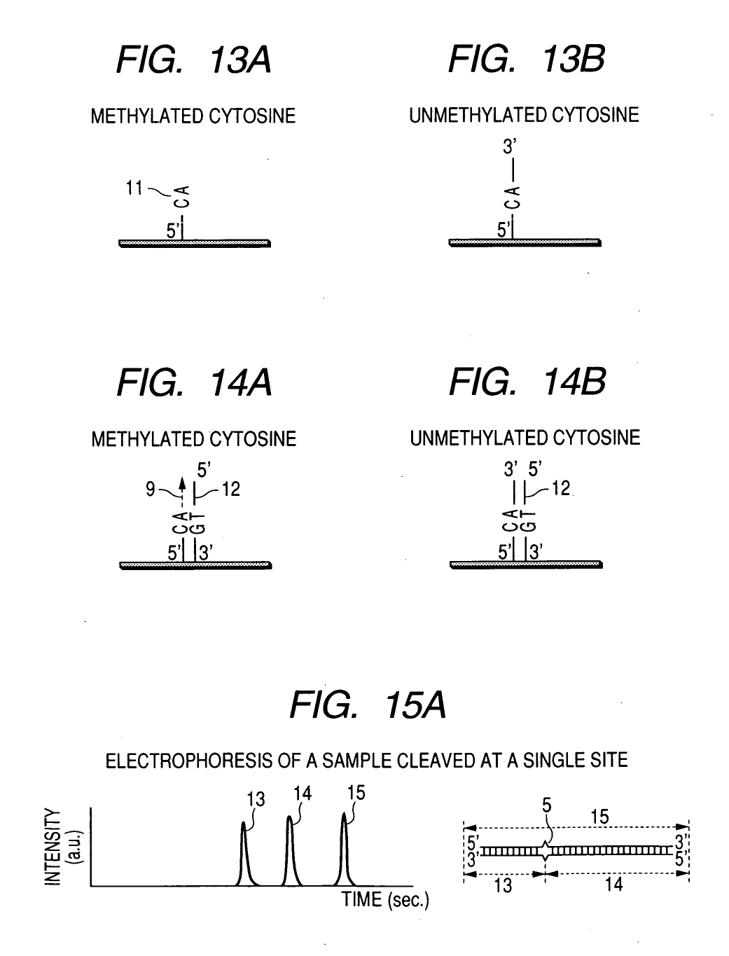

Denature solution (0.2 M NaOH) was added to the substrate to dehybridize the target sequence from the oligonucleotide immobilized on the substrate as shown in FIG. 13, and the substrate was washed twice with sterile water.

Next, 5 pmol of the oligonucleotide having the nucleotide sequence (SEQ ID NO:16) complementary to the probe was dissolved in 3 μl of PerfectHyb^{R} hybridization solution (TOYOBO Co., Ltd.) and heated for 3 min at 95 degrees C. to completely destroy the secondary structure. After allowing the solution to return to room temperature, this 3 μl was applied onto the substrate and hybridized to the probe immobilized on the substrate for 1 hour at 42 degrees C. under the condition of 100% humidity. Then, the substrate was washed twice with sterile water to remove unhybridized remnants.

Then, 0.3 μl of 10×Taq buffer (Amersham Biosciences Ltd.), 0.15 μl of 1 mM dNTP, and 0.03 μl of 5 U/μl Taq polymerase (Amersham Biosciences Ltd.) were mixed and adjusted to 3 μl with sterile water. This solution for extension reaction was applied onto the substrate, and an extension reaction was performed for 10 min at 55 degrees C. (FIG. 14).

At this time, when no methylated cytosine was present in the target sequence, the extension reaction did not proceed because the probe immobilized on the substrate was hybridized to the full length of the oligonucleotide that had been added. On the other hand, when methylated cytosines were present in the target sequence, the probe immobilized on the substrate was shortened by being digested at noncomplementary sites. Therefore, the region of the added oligonucleotide that did not hybridize to the probe immobilized on the substrate was in a single-stranded state, and the extension reaction was allowed to proceed.

After the extension reaction, the substrate was rapidly mounted on a microarray detection apparatus and added with 3 μl of the luciferin-luciferase bioluminescence reagent to detect luminescence by making use of pyrophosphate generated by the extension reaction. When no methylated cytosine was present in the target sequence, the extension reaction did not proceed and thus pyrophosphate was not produced, resulting in no observation of the luminescent reaction via a series of reactions. On the other hand, when methylated cytosines were present in the target sequence, the cleaved fragment allowed the extension reaction to proceed to produce pyrophosphate, and therefore luminescence was observed.

According to the present invention, multiple mutations in a gene, particularly methylated cytosines, can be easily detected over a wide range, thus making it possible to apply the present invention to diagnosis of diseases and the like that are linked to gene abnormalities.

What is claimed is:

1. A method for nucleic acid analysis to analyze one or more noncomplementary sites in a double-stranded nucleic acid sample, comprising the steps of:
obtaining nucleic acid fragments by cleaving the one or more noncomplementary sites with a single strand-specific endonuclease;
hybridizing at least one of the obtained nucleic acid fragments to a probe having a nucleotide sequence that is partially or totally identical to either one strand of the double-stranded nucleic acid sample;
performing an extension reaction from the nucleic acid fragment hybridized to the probe; and
optically detecting pyrophosphate generated by the extension reaction.

2. The method for nucleic acid analysis according to claim 1, further comprising the step of amplifying beforehand a sequence containing the one or more noncomplementary sites on a target nucleic acid to obtain the double-stranded nucleic acid sample.

3. The method for nucleic acid analysis according to claim 2, wherein one strand of the double-stranded nucleic acid sample is labeled with biotin in the step of amplifying and a nucleic acid fragment labeled with biotin that is generated by cleavage of the double-stranded nucleic acid with the single strand-specific endonuclease is recovered by the use of an avidin-immobilized carrier in the step of recovering nucleic acid fragments.

4. The method for nucleic acid analysis according to claim 1, wherein the probe is a probe immobilized on a solid phase carrier.

5. The method for nucleic acid analysis according to claim 2, wherein the probe is a probe immobilized on a solid phase carrier.

6. The method for nucleic acid analysis according to claim 3, wherein the probe is a probe immobilized on a solid phase carrier.

7. The method for nucleic acid analysis according to claim 1, wherein the one or more noncomplementary sites are mutation sites present in the double-stranded nucleic acid sample.

8. The method for nucleic acid analysis according to claim 7, wherein the mutation sites are methylated cytosines.

9. The method for nucleic acid analysis according to claim 8, wherein a step of treating the double-stranded nucleic acid sample with bisulfite is included prior to the step of amplifying.

10. The method for nucleic acid analysis according to claim 1, wherein the step of hybridizing is carried out by lowering the temperature at a rate not faster than 0.1 degree C./sec.

11. The method for nucleic acid analysis according to claim 1, wherein the step of optically detecting pyrophosphate is performed by a bioluminescence reaction that makes use of a luciferin-luciferase reaction.

12. The method for nucleic acid analysis according to claim 1, wherein the single strand-specific endonuclease is any one selected from CELI nuclease, mung bean nuclease, S1 nuclease, and P1 nuclease.


SEQUENCE LISTING (17 sequences)

SEQ ID NO: 1 - DNA, 26 bp, Artificial Sequence
  Description of Artificial Sequence Synthetic primer
gactgaatataaacttgtggtagttg

SEQ ID NO: 2 - DNA, 23 bp, Artificial Sequence
  Description of Artificial Sequence Synthetic primer
ctattgttggatcatattcgtcc

SEQ ID NO: 3 - DNA, 107 bp, Homo sapiens
gactgaatataaacttgtggtagttggagctggtggcgtaggcaagagtgccttgacgatacagctaattcagaatcattttgtggacgaatatgatccaacaatag

SEQ ID NO: 4 - DNA, 107 bp, Artificial Sequence
  Description of Artificial Sequence Synthetic target sequence
ctattgttggatcatattcgtccacaaaatgattctgaattagctgtatcgtcaaggcactcttgcctacgccacaagctccaactaccacaagtttatattcagtc

SEQ ID NO: 5 - DNA, 107 bp, Artificial Sequence; identical to SEQ ID NO: 3
  Description of Artificial Sequence Synthetic reference sequence

SEQ ID NO: 6 - DNA, 107 bp, Artificial Sequence
  Description of Artificial Sequence Synthetic nucleotide sequence
ctattgttggatcatattcgtccacaaaatgattctgaattagctgtatcgtcaaggcactcttgcctacgccaccagctccaactaccacaagtttatattcagtc

SEQ ID NO: 7 - DNA, 118 bp, Homo sapiens
caagcgcatatccttctaggtagcgggcagtagccgcttcagggagggacgaagagacccagcaacccacagagttgagaaatttgactggcattcaagctgtccaatcaatagctgc

SEQ ID NO: 8 - DNA, 118 bp, Artificial Sequence
  Description of Combined DNA/RNA Molecule Synthetic oligonucleotide
uaagcguatatuuttutaggtagcggguagtagucguttuagggagggacgaagagauuuaguaauuuauagagttgagaaatttgautgguattuaagutgtuuaatuaatagutgu

SEQ ID NO: 9 - DNA, 118 bp, Artificial Sequence
  Description of Combined DNA/RNA Molecule Synthetic oligonucleotide
uaaguguatatuuttutaggtaguggguagtaguuguttuagggagggaugaagagauuuaguaauuuauagagttgagaaatttgautgguattuaagutgtuuaatuaatagutgu

SEQ ID NO: 10 - DNA, 23 bp, Artificial Sequence
  Description of Artificial Sequence Synthetic primer
caaacgcatatccttctaaataa

SEQ ID NO: 11 - DNA, 26 bp, Artificial Sequence
  Description of Artificial Sequence Synthetic primer
gtagttattgattggatagtttgaat

SEQ ID NO: 12 - DNA, 118 bp, Artificial Sequence
  Description of Artificial Sequence Synthetic nucleotide sequence
caaacgcatatccttctaaataacgaacaataaccgcttcaaaaaaaaacgaaaaaacccaacaacccacaaaattaaaaaatttaactaacattcaaactatccaatcaataactac

SEQ ID NO: 13 - DNA, 118 bp, Artificial Sequence
  Description of Artificial Sequence Synthetic nucleotide sequence
caaacgcatatccttctaaataacaaacaataaccacttcaaaaaaaaacaaaaaaacccaacaacccacaaaattaaaaaatttaactaacattcaaactatccaatcaataactac

SEQ ID NO: 14 - DNA, 118 bp, Artificial Sequence
  Description of Artificial Sequence Synthetic target sequence
gtagttattgattggatagtttgaatgttagttaaattttttaattttgtgggttgttgggttttttcgtttttttttgaagcggttattgttcgttatttagaaggatatgcgtttg

SEQ ID NO: 15 - DNA, 118 bp, Artificial Sequence; identical to SEQ ID NO: 13
  Description of Artificial Sequence Synthetic reference sequence

SEQ ID NO: 16 - DNA, 118 bp, Artificial Sequence
  Description of Artificial Sequence Synthetic reference sequence
gtagttattgattggatagtttgaatgttagttaaattttttaattttgtgggttgttgggtttttttgtttttttttgaagtggttattgtttgttatttagaaggatatgcgtttg

SEQ ID NO: 17 - DNA, 118 bp, Artificial Sequence; identical to SEQ ID NO: 12
  Description of Artificial Sequence Synthetic reference sequence